(19) United States
(12) Patent Application Publication
(10) Pub. No.: US 2007/0155786 A1
(43) Pub. Date: Jul. 5, 2007

(54) Methods for treating oral aphthous stomatitis and oral mucositis

(76) Inventor: Dan Dayan, Ashkelon (IL)

Correspondence Address: Kevin D McCarthy;Roach Brown McCarthy & Gruber, 1620 Liberty Building, Buffalo, NY, 14202, US

(21) Appl. No.: 10/586,841

(22) PCT Filed: Jan. 21, 2005
(86) PCT No.: PCT/IL05/00078

§ 371 Date: Feb. 20, 2007

(30) Foreign Application Priority Data
Jan. 22, 2004 (IL) 160022
Mar. 22, 2004 (IL) 161012
Jun. 17, 2004 (IL) 162591

(51) Int. Cl. A61K 31/47; A61K 31/155; A61K 31/045

(52) U.S. Cl. 514/311; 514/312; 514/313; 514/729; 514/634

(57) ABSTRACT

The invention provides a composition comprising certain quinoline derivatives for ameliorating, treating, and preventing aphthous stomatitis and oral mucositis. The composition, optionally containing also an antiseptic, efficiently alleviates various oral conditions, such as aphthae, even at low concentrations of the active substance.

### FIELD OF THE INVENTION

The present invention relates to a composition for ameliorating, treating, and preventing oral mucosa disorders, including aphthous stomatitis, comprising a quinoline derivative.

### BACKGROUND OF THE INVENTION

Canker sores, or aphthae, are the most common oral disease which affects, in some degree, up to two thirds of the population, causing discomfort and annoyance to millions of people around the globe. The disease has unclear etiology, and it is also denoted, in various of its symptoms, as recurrent aphthous ulcers (RAU), recurrent aphthous stomatitis (RAS), or ulcerative stomatitis. RAU are characterized by repeated development of painful sores. The small, shallow and rounded ulcers develop inside the mouth, especially on the mucosa of the cheeks, lips, floor of mouth, lateral and ventral sites of the tongue, and only on rare occasions on the gums or the palate. The lesions caused by RAU may reappear at intervals of a few months to a few days. The most common presentation of the disease is minor aphthae (MiRAU) affecting about 80% of RAU patients, which is characterized by recurrent, round, clearly defined, small, painful ulcers, usually less than 5 mm in diameter with a gray-white necrotic pseudomembrane cover and a thin erythematous halo. MiRAU occur usually on the non-keratinized oral mucosa, i.e., labial and buccal mucosa as well as vestibulum and floor of mouth. These lesions may heal within 10 to 14 days without scarring. Major aphthae (MaRAU) are a less common form of the disease and are usually characterized by recurrent large ulcerations, which may be 1 to 3 cm in diameter. MaRAU occurs mainly on labial, buccal, latero-ventral mucosa of the tongue and may persist up to 6 weeks and often heal with scarring. The third and the least common clinical form of RAU are the herpetiform aphthae (HA) that is characterized by multiple (up to 100) recurrent clusters of pinpoint (2-3 mm diameter) painful ulcers, which tend to fuse, producing large areas of erosions and ulcerations. This resembles the clinical presentation of primary herpetic gingivo-stomatitis—a viral disease caused by human herpes virus type I. HA may occur on the entire oral mucosa, including keratinized mucosa, such as that of the gingiva and palate. It has a later age of onset than MiRAU and MaRAU.

From 5% to 66% of the population, depending on the group studied, are afflicted. Studies found that RAU have a tendency to recur along family lines, and a high correlation of RAU has been detected in identical twins [Miller M. F. et al.: Oral Surg. Oral Med. Oral Pathol. 43 (1977) 886-91]. Women seem to be afflicted slightly more then men. The disease seems to be less frequent, e.g., among Bedouin Arabs, but is very common in North America. Although the etiology of RAU is unknown, numerous systemic and local factors have been proposed to be involved in its pathogenesis. Among the local factors, minor trauma, such as anesthetic injections, sharp foods or trauma from dental treatment, should be considered as one of the precipitating factors of RAU. It has been suggested that oral Streptococci and several viruses may play an etiologic role in RAU; however, no conclusive results have been achieved. The involvement of inflammatory cytokines in RAU was implicated [Buno I. J. et al.: Arch. Dermatol. 134 (1998) 827-31]. RAU was also associated with immune disturbances [Eversole L. R. Oral Surg. Oral Med. Oral Pathol. 77 (1994) 555-71]. The systemic and local cellular immunodisregulation associated with RAU seems to be consistent with a viral reactivation, and may be a result of a latent viral infection of oral mucosa [Pedersen A. et al.: Oral Pathol. Med. 22 (1993) 64-8]. RAU was also observed in several systemic disorders, such as Behcet's disease, cyclic neutropenia, MAGIC syndrome, FAPA syndrome, celiac disease, inflammatory bowel disease, HIV, ulcus vulvae acuthum, and hemato-deficiencies, such as iron, zinc, and vitamin deficiencies [Ship J.: Oral Surg. Oral Med. Oral Pathol. Oral Radiol. Endod. 81 (1996) 141-7]. There is no specific treatment for RAU, and the management usually depends on the symptoms, duration and severity of the ulcerative lesions. In cases of RAU resulting from a systemic disorder, the corresponding therapy for said disorder can be efficient, for example in cases of a nutritional or vitamin deficiency, a replacement therapy is used.

Despite detailed clinical and research investigation over the years, the causes of the disease, first described by Hippocrates, are still unknown, and no effective management is available for it. Therefore, a need is felt for new means that could either heal aphthae or at least ameliorate them more efficiently.

The disorders of mouth mucosa, being manifested by vesicular-bullous ulcerative, or erosive, lesions, may be diagnosed also as chronic discoid lupus erythematosus, herpetiform dermatitis, pemphigus family disorders, pemphigoid family disorders, linear IgA disorders or other immunoregulatory disorders, and similarly to the aphthae family, their exact etiology is unclear; they are quite common—but less frequent than aphthae. To this greater family of mouth mucosa disorders, radiotherapeutic mucositis and chemotherapeutic mucositis, the conditions caused by radiotherapy and chemotherapy, may be added, even though in this case, etiology is clearer, and involves cytotoxic effects of said therapies. What is common to all the mentioned oral mucosa disorders, including aphthae related disorders, is the lack of knowledge regarding the precise mechanism by which they develop, and the lack of efficient therapies for these painful conditions. All said mouth mucosa disorders will be called oral mucositis hereinafter.

The most common treatment for oral mucositis of various origin is topical therapy, which may include antimicrobial and analgesic mouthwashes, topical or systemic glucocorticoids, immunosuppressors and hormones. The most common topical therapy is the use of hydrocortisone, triamcinolone, fluocinonide, betamethasone and flumethasone [Scully C. et al.: J. Oral Pathol. Med. 18 (1989) 21-7]. Immunosuppressive drugs, such as colchicine, cyclosporin and thalidomide, as well as immunopotentiating agents, such as levamisole, gammaglobulin and longovital were also tried without clear results. Some topical medications seemed to have certain beneficial effects on the ulcers of RAU, such as sucralfate [Ratan J. et al.: J. Int. Med. 236 (1994) 341-3], azelastine hydrochloride [Ueta E. et al.: J. Oral Pathol. Med. 23 (1994) 123-9], prostaglandin E2 [Taylor L. J. et al.: Br. Dent. J. 175 (1993) 125-9], listerine [Meiller T. F. et al.: Oral Surg. Oral Med. Oral Pathol. 72 (1991) 425-9], diclofenac in hyaluronan [Saxen M. A. et al.: Oral Surg. Oral Med. Oral Pathol. Oral Radiol. Endod. 84 (1997) 356-61], or bioadhesive hydrogel patches [Mahdi A. B. et al.: J. Oral Pathol. Med. 25 (1996) 416-9]. All said treatments are palliative, reducing the symptom manifestation. U.S. Pat. No. 5,686,095 discloses a method for topically treating aphthous ulcerations, comprising fluoroquinolone derivatives.

It is therefore an object of this invention to provide an efficacious pharmaceutical composition for use in ameliorating, treating, and preventing oral aphthous stomatitis and oral mucositis.

It is further an object of this invention to provide an efficacious pharmaceutical composition for accelerated healing of aphthae, and for mitigating pains caused by them.

It is another object of this invention to provide an efficacious pharmaceutical composition for treating aphthous stomatitis and oral mucositis when secondary complications occur.

It is still another object of the invention to provide an efficacious composition for aphthae prevention.

It is a still further object of this invention to provide compositions for treating aphthous stomatitis and oral mucositis with low concentrations of quinoline derivatives.

Other objects and advantages of present invention will appear as description proceeds.

### SUMMARY OF THE INVENTION

The present invention provides a pharmaceutical composition for ameliorating, treating, and preventing oral mucosa disorders, including canker sores associated with aphtha minor, aphtha major, recurrent aphthous ulcers (RAU), recurrent aphthous stomatitis (RAS), herpetiform aphthae, vesicular-bullous ulcerative or erosive lesions, pemphigus family disorders, pemphigoid family disorders, linear IgA disorders or other immunoregulatory disorders, herpetiform dermatitis, chronic discoid lupus erythematosus, radiotherapeutic mucositis, and chemotherapeutic mucositis, wherein said disorders may be complicated by secondary infections, comprising a first component and optionally a second component wherein said two components may be applied simultaneously or subsequently, said second component being an antiseptic and said first component being quinoline derivative of formula I: 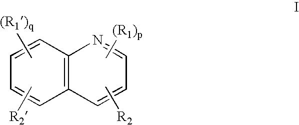
or its stereoisomer, or its pharmaceutically acceptable salt, wherein R₁ and R₁′ are independently selected from —H, —Cl, —F, C₁-C₃ alkyl, C₁-C₃ alkyloxy, and —CF₃; R₂ and R₂′ are independently selected from —H, —NH(R₃), and —C(OH)(R₃), wherein R₃ is selected from phenyl and C₃-C₆ alkyl, substituted with 1 to 3 substituents selected from C₁-C₂ alkyl, ethenyl, —OH, and —NH₂, and wherein said —NH₂ is either optionally substituted with one or two groups selected from ethyl and hydroxyethyl or the nitrogen atom of said —NH₂ is connected with 1 or 2 carbon atoms of said C₃-C₆ alkyl or C₁-C₂ alkyl, possibly forming bicyclic structure; p is an integer from 1 to 3, and q is an integer from 1 to 4. Said antiseptic is selected from acceptable antiseptics known in the art, such as chlorhexidine, thymol, esters of p-hydroxybenzoic acid, etc.

Any of the two components of said optionally two-component composition may further comprise a constituent selected from solvents, buffers, carriers, binding agents, stabilizers, adjuvants, diluents, excipients, surfactants, and odorants, as well as another pharmaceutically active substance selected from analgesic, anti-inflammatory, antiviral, antibacterial, antifungal, and antineoplastic.

This invention also relates to a method for treating, ameliorating, and preventing conditions comprising the appearance on the oral mucosa of painful sores, vesicles, bullae, ulcers, erosions, lesions, or blisters, associated for example with aphthae, discoid lupus erythematosus, pemphigus, pemphigoid, herpetiform dermatitis, radiotherapy and chemotherapy, wherein said conditions may be accompanied by a secondary infection, comprising the steps of i) providing a quinoline derivative of formula I as defined above, or its stereoisomer or a pharmaceutically acceptable salt thereof; optionally ii) providing an antiseptic; iii) preparing a one-component formulation comprising said quinoline derivative; or alternatively preparing two-component composition comprising either two formulations separately containing said quinoline derivative (or its isomer or salt) and said antiseptic, or one formulation containing a mixture of said antiseptic and quinoline derivative (or its isomer or salt) in one solution or suspension; wherein any of said formulations may further comprise constituents adjusting the consistency, stability, and olfactory properties, and optionally additional active substances; and iv) administering said formulation(s) to a patient in need of the treatment; wherein said two components may be administered simultaneously or subsequently. Said administration of said formulation(s) comprises rinsing, spraying, and applying ointment or adhesive patch. Said administration may comprise rinsing with said formulation(s) and swallowing the formulation not containing said antiseptic. In a preferred method according to this invention, a mucosa disorder associated with aphtha is treated by rinsing mouth several times a day with a liquid comprising said quinoline derivative, and several times a day with a liquid comprising an antiseptic, selected, e.g., from a esters of p-hydroxybenzoic acid, thymol, and chlorhexidine.

In a preferred embodiment of the invention, said pharmaceutical composition, or said pharmaceutical formulation, comprises said quinoline derivative in a concentration of from 0.04 mg/ml to 10 mg/ml. In another preferred embodiment, said pharmaceutical composition, or said pharmaceutical formulation, comprises said quinoline derivative in a concentration of from 0.050 mg/ml to 0.120 mg/ml.

### DETAILED DESCRIPTION OF THE INVENTION

It has now been found that some quinoline derivatives are surprisingly effective in treating oral aphthous stomatitis and oral mucositis. In experiments carried out with particular preferred compounds of the invention, the pain caused by ulcerative lesions disappeared often within hours, and the ulcers and other symptoms disappeared usually within days. For example, patients with life-long recurrent aphthae problems achieved quick relief of pains caused by aphthae ulcers after rinsing their mouth with aqueous solutions comprising said compounds. The effect is often enhanced when said quinoline derivative is used together with an antiseptic.

It has been further found that oral aphthous stomatitis and oral mucositis are efficiently healed even when using surprisingly low concentrations of quinoline derivatives of formula I. For example, patients with major aphtha accompanied by a secondary infection achieved relief of pains and healing through rinsing their mouth with a composition comprising 0.083 mg/ml of quinine, and optionally chlorhexidine, each rinsing performed for several minutes, several times a day. Quinine at levels up to 83 ppm is approved by FDA for use in beverages as a flavoring agent.

Aphthous stomatitis and oral mucositis is often accompanied by a secondary infection, which can aggravate the symptoms and complicate the treatment. The composition according to the invention is useful also for these complicated cases, offering a synergistic effect provided by the two-component system comprising a quinoline derivative of formula I and an antiseptic.

Whenever the term “two-component composition” is used herein, a system is meant that comprises at least one antiseptic and at least one quinoline derivative of formula I, wherein the two components may be mixed, forming a homogeneous formulation to be applied simultaneously, or alternatively the two components may be separated in two formulations and applied subsequently in any order. It is understood that said term does not exclude the presence of other components (constituents).

In a preferred embodiment of this invention, a two-component composition comprising an antiseptic selected from accepted antiseptics, such as chlorhexidine or esters of p-hydroxybenzoic acid, and a quinoline derivative of formula I:

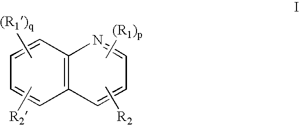

or a stereoisomer thereof, or a pharmaceutically acceptable salt thereof, wherein

R₁ and R₁′ are independently selected from —H, —Cl, —F, C₁-C₃ alkyl, C₁-C₃ alkyloxy, and —CF₃; R₂ and R₂′ are independently selected from —H, —NH(R₃), and —C(OH)(R₃), wherein R₃ is selected from phenyl and C₃-C₆ alkyl, substituted with 1 to 3 substituents selected from C₁-C₂ alkyl, ethenyl, —OH, and —NH₂, and wherein said —NH₂ is either optionally substituted with one or two groups selected from ethyl and hydroxyethyl, or the nitrogen atom of said —NH₂ is connected with 1 or 2 carbon atoms of said C₃-C₆ alkyl or C₁-C₂ alkyl, forming secondary or tertiary amine, possibly forming bicyclic structure; p is an integer from 1 to 3; and q is an integer from 1 to 4; is applied on oral mucosa afflicted with sores, ulcers, erosions, vesicular-bullous lesions, blisters, stria, or other painful changes, in form of a solution, suspension, gel, emulsion, ointment, patch, or spray; wherein both components are applied simultaneously or subsequently. When the components are applied separately, the number of treatments performed daily with said quinine derivative and with said antiseptic need not be the same (for example, three quinine mouth washes may be interspersed with two chlorhexidine washes, etc.). Said solution and suspension are preferably based on aqueous solutions of pharmaceutically acceptable salts and buffers, such as physiological solution, etc, but may contain acceptable non-aqueous solvents, such as ethanol, DMSO, etc. Said gel, emulsion, or ointment may comprise pharmaceutically acceptable oils and surfactants, and are prepared by methods known in the art of topical formulations, therefore not requiring detailed descriptions for their preparations. Said active agent of formula I may be either dissolved in at least one phase of the composition, or may be partially dispersed. Said ester of p-hydroxybenzoic acid may be selected from methyl, ethyl, propyl, and butyl.

The composition of the invention is applied preferably on the oral mucosa afflicted with a disorder selected from canker sores associated with aphtha minor, aphtha major, recurrent aphthous ulcers (RAU), recurrent aphthous stomatitis (RAS), herpetiform aphthae, vesicular-bullous erosive or ulcerative lesions, pemphigus family disorders, pemphigoid family disorders (e.g. cicatricial), linear IgA disorders or other immunoregulatory disorders, herpetiform dermatitis, discoid lupus erythematosus, radiotherapeutic mucositis, or chemotherapeutic mucositis. In a preferred embodiment of this invention, the two-component composition is applied on oral mucosa further afflicted by an accompanying infection.

In a preferred embodiment of the invention, the two-component composition for mitigating and healing symptoms of oral mucositis comprises an antiseptic and a quinoline derivative of formula I: 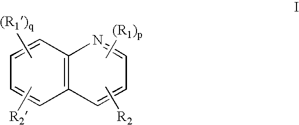
wherein R₁ and R¹′ are independently selected from —Cl, —OCH₃, and —CF₃; one of R₂ and R₂′ is —H and the other is selected from —NH(R₃), and —C(OH)(R₃), wherein R₃ is selected from phenyl and C₃-C₅ alkyl, substituted with 1 to 2 substituents selected from C₁-C₂ alkyl, ethenyl, and —NH₂, and wherein either said —NH₂ is optionally substituted with one or two groups selected from ethyl and hydroxyethyl, or the nitrogen atom of said —NH₂ is connected with 1 or 2 carbon atoms of said C₃-C₅ alkyl or C₁-C₂ alkyl, possibly forming bicyclic structure; and wherein the sum of p and q is an integer from 1 to 3; wherein said two components may be applied simultaneously or subsequently. Said quinoline derivative has preferably a concentration of from 0.04 mg/ml to 10 mg/ml, and more preferably a concentration of from 0.05 mg/ml to 0.120 mg/ml.

In a preferred embodiment of the invention, the composition for ameliorating or treating or preventing oral mucositis comprises a quinoline derivative of formula II and optionally an antiseptic: 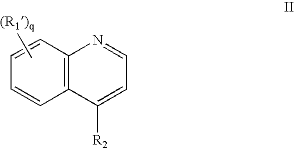
wherein

R₁′ is selected from —Cl, C₁-C₃ alkyloxy, and —CF₃; R₂ is selected from —NH(R₃), and —C(OH)(R₃), wherein R₃ is C₃-C₆ alkyl substituted with 1 to 3 substituents selected from C₁-C₂ alkyl, ethenyl, and —NH₂, and wherein said —NH₂ is either optionally substituted with one or two groups selected from ethyl and hydroxyethyl or the nitrogen atom of said —NH₂ is connected with 1 or 2 carbon atoms of said C₃-C₆ alkyl or C₁C₂ alkyl, possibly forming bicyclic structure, and wherein q is 1 or 2; wherein the two components may be applied simultaneously or subsequently. Said quinoline derivative may have a concentration of from 0.050 mg/ml to 0.085 mg/ml.

In a preferred embodiment of this invention, oral mucositis is treated by a two-component composition comprising solutions or dispersions comprising hydroxychloroquine (HCQ) or its salt, and chlorhexidine. Preferably HCQ salt is dissolved in an aqueous buffered solution, and used several times a day for rinsing mouth without swallowing, followed by rinsing with chlorhexidine solution. The concentration of HCQ or its salt in said solution is preferably about 0.083 mg/ml, and the rinsing is preferably performed 3-5 times a day. HCQ is preferably used as sulfate. Chlorhexidine solution has preferably a concentration from 0.05% to 0.2%.

In other preferred embodiment of this invention, oral mucositis is treated by a quinine (Q) salt in solution or suspension, which is used several times a day for rinsing mouth without swallowing, followed by rinsing with chlorhexidine solution. The concentration of Q is preferably about 0.083 mg/ml, and chlorhexidine solution has preferably a concentration from 0.05% to 0.2%. The rinsing with each of the two components is preferably performed 3-5 times a day. Said Q solution may comprise Q dihydrochloride, Q hydrochloride, Q sulfate, and other Q salts. Said Q suspension may, for example, comprise Q sulfate. Stereoisomers of quinine and their salts, such as quinidine sulfate, have been also found active.

In still another preferred embodiment of this invention, oral mucositis is treated several times a day by a quinine (Q) salt in solution or suspension which is swallowed after rinsing mouth, followed by rinsing with a chlorhexidine solution. The concentration of Q is preferably about 0.083 mg/ml, and said chlorhexidine solution (which is not swallowed) has preferably a concentration from 0.05% to 0.2%.

The invention provides a method for treating a symptom associated with oral mucositis selected from canker sores associated with aphtha minor, aphtha major, recurrent aphthous ulcers (RAU), recurrent aphthous stomatitis (RAS), herpetiform aphthae, bullous erosive or ulcerative lesions, pemphigus disorders, pemphigoid disorders, linear IgA disorders and other immunoregulatory disorders, chemotherapeutic mucositis, and radiotherapeutic mucositis, comprising preparing a two-component-composition, one component being an antiseptic and the other one a quinoline derivative of formula I or a pharmaceutically acceptable salt thereof, in form of paste, cream, gel, patch impregnated with an active agent, or spray, and applying said composition onto the painful areas or areas afflicted with the pathological changes, wherein the application of the two components may be simultaneous or subsequent. Other pharmaceutically effective agents may be present in the composition of the invention, to enhance the healing process, and taking into account eventual other disorders involved. In one preferred embodiment, the method of the invention comprises an adhesive patch impregnated with a two-component composition containing an antiseptic and a quinoline derivative of formula I, which patch is placed on the inflicted areas repeatedly, until the pain and other aphthae symptoms disappear. In another preferred embodiment an adhesive patch may comprise only the quinoline component of the two-component composition, and the antiseptic component is applied through subsequent rinsing. In still another preferred embodiment, mouth rinsing with a liquid composition according to this invention is performed several times a day.

In a preferred use according to this invention, a composition comprising an antiseptic and a quinoline derivative, preferably selected from HCQ, quinine, their isomers and derivatives, and their salts, prolong the recurrence periods in persons suffering from RAU. Applying said two-component composition, either through simultaneous presence of both components or through their subsequent use, ameliorates the symptoms, heals ulcers, and typically also prevents the reappearance of new ulcers.

A composition according to the invention may comprise a constituent selected from solvents, buffers, carriers, binding agents, stabilizers, adjuvants, diluents, excipients, surfactants, flavors, and odorants. In a preferred embodiment of this invention, the composition for treating oral aphthous stomatitis and oral mucositis, comprises another pharmaceutically active substance selected from analgesic, anti-inflammatory, antiviral, antibacterial, antifungal, and antineoplastic compounds. In a preferred embodiment said solvents comprise an aqueous solution. Non-aqueous solvents may be used in the composition of the invention, not only to adjust the required consistency of the composition or the solubility of the components, but also to synergistically improve the efficiency of the composition.

In a preferred embodiment of this invention, the composition is applied topically, by rinsing the afflicted area or washing the mouth, or alternatively spraying the afflicted area. The time of action of the healing composition of this invention is prolonged when an adhesive patch impregnated with said two-component composition, or with one of its components, is applied onto the afflicted area.

The invention will be further described and illustrated in the following examples.

### EXAMPLES

### Materials

Hydroxychloroquine (HCQ) was obtained from Sanofi-Syntelabo, Inc. New York USA, quinine (Q) and quinidine sulfate were obtained from Rakah, Holon, Israel. The compounds were mixed with tap water. Chlorhexidine was obtained from LACER S.A., Barcelona, Spain.

### Example 1

A 40-years-old woman, generally in good health, reported the presence of canker sores for several years (periodic recurrences of 3 to 4 episodes per year). At clinical examination she had a minor aphtha on the vestibular mucosa on the right side of the lower jaw adjacent to teeth #45-46-47. The patient was seen five days after the appearance of the painful aphtha. She started oral rinses with 10 ml of hydroxychloroquine (HCQ) solution, 3.7 mg/ml, three times a day, each rinse for 3 to 5 min. Twenty-four hours after the start of the treatment she was free of any pain, and the aphtha almost disappeared. She continued to rinse for about a week. No additional aphthous lesions appeared during 8 weeks of follow-up.

### Example 2

A 30-years-old woman, in otherwise general good health, came with two painful major aphthae, one of 12 mm diameter on the left side of the lower lip mucosa and the second on the right latero-ventral side of the tongue, about 8 mm diameter. She reported the presence of recurrent aphthous lesions since she was about 8-years-old. During the last year, she continuously suffered from recurrent episodes of major aphthae, with virtually no aphtae-free intervals. Each episode of aphthae usually lasted for 3 to 6 weeks. She started oral rinses with 10 ml of HCQ solution, 3.7 mg/ml, three times a day, each rinse for 3 to 5 min. At clinical examination 5 days later, she reported that the pain completely disappeared 24 hours after the start of rinses. The major aphtha of the lower lip decreased in size to about half its original size and the aphtha of the tongue completely disappeared. At clinical examination after 6 additional days, the major aphtha on the lower lip mucosa disappeared completely, leaving a pronounced scar. Concomitantly, 2 new lesions of minor aphthae, 3 mm in diameter each, were seen, one on the left side of the upper lip mucosa and the other on the ventro-anterior side of the tongue. After 4 more days of rinsing with the same HCQ solution, the aphthous lesion on the tongue disappeared, and that on the lip diminished significantly in its size. These two aphthous lesions did not cause any pain whatsoever. In total, she rinsed with the HCQ solution for 20 days. Follow up for 3 months revealed that no new aphthous lesions were present. The patient reported that this was the longest aphthae-free interval that she remembered during the last years and that her psychological state has significantly improved.

### Example 3

A 10-years-old girl, in otherwise general good health, reported the presence of recurrent aphthous lesions of different sizes for the last 2 years. The last episode had been about 3 months before examination. The lesions had disappeared within 2 weeks after using different conventional treatments. At clinical examination, one aphtha of about 3 mm diameter was found on the lower lip mucosa and another one about 6 mm diameter was found on the upper lip mucosa. Both lesions were painful. She started rinsing with 10 ml of HCQ solution, 2.5 mg/ml, three times a day, each rinse for 3 to 5 min. Examination after 5 days revealed that the lesion on upper lip almost disappeared leaving an erythematous area, and the aphtha on the lower lip decreased to about 1 mm diameter. She reported to be free of any pain already after 24 hours from the start of rinsing. She, therefore, was able to eat everything. The patient was advised to continue rinsing with 10 ml of hydroxychloroquine (HCQ) solution, 3.7 mg/ml, three times a day, each rinse for 3 to 5 min for 7 additional days. Within 2 days she reported that the aphtha on the lower lip disappeared completely. She still continued with the rinses for 5 additional days, as recommended. The follow up of 5 weeks after cessation of the rinses did not reveal any new aphthae.

### Example 4

A 50-years-old male reported suffering from recurrent episodes of canker sores for many years, with 2 to 3 episodes per year. The last episode had occurred about 6 months before examination. He was diagnosed with diabetes mellitus, so that use of steroidal modalities for treatment of his aphthous lesions was contraindicated. At examination he had a painful major aphthous lesion of about 8 mm diameter at the border of the left lateral side of the tongue and floor of the mouth. He started rinsing with 10 ml of HCQ solution, 1.2 mg/ml, three times a day, each rinse for 3 to 5 min. After 24 hours from start of rinses he was free of any pain and was able to eat everything. After 7 days of rinsing, the aphtha almost disappeared. He remained aphthae-free for 7 months.

### Example 5

A 23-years-old man, generally in good health, reports the presence of canker sores since childhood with about 8 to 10 recurrences per year. At clinical examination a minor painful aphtha was present on the mucosa of the lower lip. The ulcer was about 4-5 mm in diameter and was present for 8 days. Common treatment with different ointments did not bring much relief. He started to rinse with 10 ml of hydroxychloroquine (HCQ) solution, 1.2 mg/ml, three times a day, each rinse for 3 to 5 min. After one day of rinses he reported no pain and after three days he reported that the aphtha completely disappeared. The follow up of 4 months revealed no new aphthae.

### Example 6

A 36-year-old woman presented a painful minor aphtha of about 3 mm in diameter in the right vestibulum of the lower jaw adjacent to the first molar. She reported the presence of pain and inconvenience for 2 days. In the past, she had experienced recurrences of such lesions every 3 to 4 months. She started to rinse with 10 ml of 0.085 mg/ml quinine solution for 3 to 5 minutes, about 5 to 7 times a day. Pain and inconvenience disappeared after 2 days and the ulcer healed completely within 4 days.

### Example 7

A 55-year-old woman presented diffuse erosions/ulcerations of the buccal, labial and lingual mucosae that were present for at least 6 months. She complained of pain and difficulty to eat and speak. The erosive/ulcerative lesions were clinically specific for erosive lichen planus. She started to rinse (for 5 to 10 min) and swallow about 10- 20 ml of 0.085 mg/ml quinine solution, 5 to 7 times a day. After 3 days, there was a significant amelioration of her troubles, in respect to both pain and masticatory function. After about 4 weeks, during which she followed the above mentioned protocol, most of her erosive/ulcerative lesions showed marked healing and became completely asymptomatic. She did not remember such an improvement in her condition when she had been treated by means of both topical (rinses) and systemic (tablets) steroids. She was able to maintain a stable asymptomatic condition of her lichen planus by means of quinine solution during about 1 year follow-up period.

### Example 8

A 50-year old woman, generally in good health, presented a most painful, distressing major aphtha of approximately 1 cm diameter on the ventro-lateral tongue. She had suffered the presence of such lesions for several years (recurrences of 6 to 8 episodes per year). The current lesion had been present for about one week. During examination 2 lymph nodes were palpated in the floor of mouth, probably due to a secondary infection. She started to apply a rinse with 3.72 mg/cc HCQ solution (for 3 to 5 minutes) followed by a rinse with 0.12% chlorhexidine alcohol free solution (for about 1 minute), 3 times a day. Two days later, the patient was totally free of pain and the size of the aphtha diminished to about 7 mm diameter. After 2 additional days of treatment, the aphtha completely disappeared.

### Example 9

A 37-year old male, presented a painful minor aphtha on the ventro-lateral part of the tongue. He reported its appearance about 1 week ago. In the past, frequent episodes of minor aphthae had occurred every 2 months. He started to rinse with 10 ml of quinine sulfate Solution 3 mg/cc (for 3 to 5 minutes) followed by a rinse with 0.12% chlorhexidine alcohol free solution (for about 1 minute), 3 times a day. The pain decreased significantly within 24 hours, and all the inconvenience disappeared within 48 hours.

### Example 10

A 38-year-old man, in general good health, presented a minor aphtha on the lower labial mucosa of about 4 mm in diameter. He reported pain and discomfort for about 3 days. He had suffered in the past of similar lesions with a frequency of about one episode for every 2 to 3 months. He started rinsing with 10 ml of 0.085 mg/ml quinine solution, about 5 times per day, each time for about 3 to 5 min. After each rinse he swallowed the solution. He became free of any symptoms within 24 hours and reported the disappearance of the aphtha in about 4 days. Follow-up of about 6 months revealed no further recurrences.

While this invention has been described in terms of some specific examples, many modifications and variations are possible. It is therefore understood that within the scope of the appended claims, the invention may be realized otherwise than as specifically described.

1-27. (canceled)

28. A quinoline derivative of formula I: 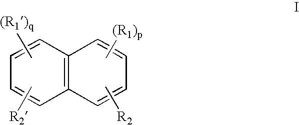
or a pharmaceutically acceptable salt thereof, wherein
R₁ and R₁′ are independently selected from —H, —Cl, —F, C₁-C₃ alkyl, C₁-C₃ alkyloxy, and —CF₃;
R₂ and R₂′ are independently selected from —H, —NH(R₃), and —C(OH)(R₃), wherein R₃ is selected from phenyl and C₃-C₆ alkyl, substituted with 1 to 3 substituents selected from C₁-C₂ alkyl, ethenyl, —OH, and —NH₂, and wherein said —NH₂ is either optionally substituted with one or two groups selected from ethyl and hydroxyethyl, or the nitrogen atom of said —NH₂ is connected with 1 or 2 carbon atoms of said C₃-C₆ alkyl or C₁-C₂ alkyl, possibly forming bicyclic structure;
p is an integer from 1 to 3; and q is an integer from 1 to 4; and an antiseptic for combined use in ameliorating, treating, and preventing aphthous stomatitis and oral mucositis.

29. A quinoline derivative and an antiseptic according to claim 28, wherein said antiseptic is selected from the group consisting of chlorhexidine, thymol, and esters of p-hydroxybenzoic acid selected from methyl, ethyl, propyl, and butyl.

30. A quinoline derivative and an antiseptic according to claim 28, further comprising a constituent selected from solvents, buffers, carriers, binding agents, stabilizers, adjuvants, diluents, excipients, surfactants, flavors, and odorants.

31. A quinoline derivative and an antiseptic according to claim 28, further comprising another pharmaceutically active substance selected from analgesic, anti-inflammatory, antiviral, antibacterial, antifungal, antiseptic, and antineoplastic compounds.

32. A quinoline derivative and an antiseptic c according to claim 28, wherein said antiseptic and quinoline derivative are applied subsequently, in any order.

33. A quinoline derivative and an antiseptic according to claim 28, wherein said antiseptic and quinoline derivative are applied simultaneously.

34. A quinoline derivative and an antiseptic according to claim 28, for topical use.

35. A quinoline derivative and an antiseptic according to claim 28, for oral delivery.

36. A quinoline derivative and an antiseptic according to claim 34, wherein said use comprises rinsing with liquid, or applying cream, ointment, gel, patch, or spray.

37. A quinoline derivative and an antiseptic according to claim 28, wherein said stomatitis or mucositis comprises canker sores associated with aphtha minor, aphtha major, recurrent aphthous ulcers (RAU), recurrent aphthous stomatitis (RAS), herpetiform aphthae, vesicular-bullous erosive or ulcerative lesions, pemphigus family disorders, pemphigoid family disorders, linear IgA disorders or other immunoregulatory disorders, herpetiform dermatitis, discoid lupus erythematosus, radiotherapeutic mucositis, or chemotherapeutic mucositis.

38. A quinoline derivative and an antiseptic according to claim 37, wherein said mucositis or stomatitis is accompanied by a secondary infection.

39. A quinoline derivative and an antiseptic according to claim 28, wherein in said quinoline derivative of formula I, as defined in claim 28,
R₁ and R₁′ are independently selected from —Cl, —OCH₃, and —CF₃; one of R₂ and R₂′ is —H, and one of R₂ and R₂′ is selected from —NH(R₃), and —C(OH)(R₃), wherein R₃ is selected from phenyl and C₃-C₅ alkyl, substituted with 1 to 2 substituents selected from C₁-C₂ alkyl, ethenyl, and —NH₂, and wherein either said —NH₂ is optionally substituted with one or two groups selected from ethyl and hydroxyethyl, or the nitrogen atom of said —NH₂ is connected with 1 or 2 carbon atoms of said C₃-C₅ alkyl or C₁-C₂ alkyl, possibly forming bicyclic structure; and the sum of p and q is an integer from 1 to 3.

40. A quinoline derivative and an antiseptic according to claim 28, wherein said quinoline derivative has formula II: 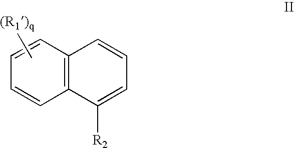
wherein
R₁′ is selected from —Cl, C₁-C₃ alkyloxy, and —CF₃;
R₂ is selected from —NH(R₃), and —C(OH)(R₃), wherein R₃ is C₃-C₆ alkyl substituted with 1 to 3 substituents selected from C₁-C₂ alkyl, ethenyl, and —NH₂, and wherein said —NH₂ is either optionally substituted with one or two groups selected from ethyl and hydroxyethyl or the nitrogen atom of said —NH₂ is connected with 1 or 2 carbon atoms of said C₃-C₆ alkyl or C₁-C₂ alkyl, possibly forming bicyclic structure, and q is 1 or 2.

41. A quinoline derivative and an antiseptic according to claim 28, comprising a stereoisomer, or a mixture of stereoisomers, of a quinoline derivative according to claim 28.

42. A quinoline derivative and an antiseptic according to claim 41, wherein the compound of formula I is selected from quinine, quinidine, hydroxychloroquine, and a salt thereof.

43. A quinoline derivative and an antiseptic according to claim 40, wherein said mucositis comprises canker sores associated with aphtha minor, aphtha major, recurrent aphthous ulcers, or recurrent aphthous stomatitis.

44. A quinoline derivative and an antiseptic according to claim 43, wherein said mucositis is accompanied by a secondary infection.

45. A quinoline derivative and an antiseptic according to claim 28, wherein said quinoline derivative or said pharmaceutically acceptable salt thereof has a concentration of from 0.04 mg/ml to 10 mg/ml.

46. A quinoline derivative and an antiseptic according to claim 45, wherein said quinoline derivative or said pharmaceutically acceptable salt thereof has a concentration of from 0.05 mg/ml to 0.120 mg/ml.

47. A method for ameliorating, treating, and preventing an oral mucosa disorder, comprising
i) providing a quinoline derivative of formula I: 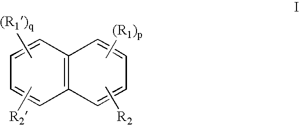
or a stereoisomer thereof or a pharmaceutically acceptable salt thereof, wherein
R₁ and R₁′ are independently selected from —H, —Cl, —F, C₁-C₃ alkyl, C₁-C₃ alkyloxy, and —CF₃;
R₂ and R₂′ are independently selected from —H, —NH(R₃), and —C(OH)(R₃), wherein R₃ is selected from phenyl and C₃-C₆ alkyl, substituted with 1 to 3 substituents selected from C₁-C₂ alkyl, ethenyl, —OH, and —NH₂, and wherein said —NH₂ is either optionally substituted with one or two groups selected from ethyl and hydroxyethyl, or the nitrogen atom of said —NH₂ is connected with 1 or 2 carbon atoms of said C₃-C₆ alkyl or C₁-C₂ alkyl, possibly forming bicyclic structure;
p is an integer from 1 to 3; and q is an integer from 1 to 4;
ii) providing an antiseptic;
iii) preparing a two-component composition comprising either two formulations containing separately said antiseptic and said quinoline derivative (or its isomer or salt), or one formulation comprising a mixture of said antiseptic and quinoline derivative in solution or suspension; wherein said formulations may further comprise constituents adjusting the consistency, stability, and olfactory properties, and optionally an additional active substances selected from analgesic, anti-inflammatory, antiviral, antibacterial, antifungal, antiseptic, and antineoplastic; and
iv) administering said formulation or formulations to a patient in need of the treatment, wherein the two components in said two-component composition may be administered simultaneously or subsequently.
v) The method of claim 47, wherein said administration of said formulation or formulations comprises rinsing, spraying, and applying ointment or adhesive patch.

48. The method of claim 47, wherein said administration comprises rinsing with said formulation or formulations, and swallowing at least one of the formulations.

49. The method of claim 47, wherein said mucosa disorder is associated with aphtha, and wherein said administration comprises rinsing mouth several times a day.

50. The method of claim 48, wherein said rinsing comprises two liquids, one comprising an antiseptic, and the other a compound of formula I.

51. The method of claim 47, wherein said antiseptic is chlorhexidine in an alcohol-free water solution.

52. The method of claim 47, wherein said compound of formula I is selected from quinine, quinidine, hydroxychloroquine, and a salt thereof.
